(19) United States
(12) Patent Application Publication
(10) Pub. No.: US 2010/0120016 A1
(43) Pub. Date: May 13, 2010

(54) METHODS AND SYSTEMS FOR DETECTION OF CONTAMINANTS

(76) Inventors: Yanbin Li, Fayetteville, AR (US); Billy Hargis, Fayetteville, AR (US); Steve Tung, Fayetteville, AR (US); Luc Berghman, Colege Station, TX (US); Walter Bottje, Fayetteville, AR (US); Ronghui Wang, Fayetteville, AR (US); Zunzhong Ye, Fayetteville, AR (US); Madhukar Varshney, Fayetteville, AR (US); Balaji Srinivasan, Fayetteville, AR (US)

Correspondence Address: MICHAEL BEST & FRIEDRICH LLP, Two Prudential Plaza, 180 North Stetson Avenue, Suite 2000, CHICAGO, IL, 60601, US

(21) Appl. No.: 12/439,462

(22) PCT Filed: Aug. 31, 2007
(86) PCT No.: PCT/US07/77376

§ 371 Date: Dec. 2, 2009

Related U.S. Application Data
(60) Provisional application No. 60/841,774, filed on Sep. 1, 2006.
(60) Provisional application No. 60/876,919, filed on Dec. 22, 2006.

(51) Int. Cl. C12Q 1/70

(52) U.S. Cl. 435/5

(57) ABSTRACT

An impedance biosensor for detecting a contaminant in a starting material, the biosensor comprising a housing, an input device supported by the housing, an output device supported by the housing, a microfluidic cell supported by the housing, the starting material being engagable with the microfluidic cell, and an impedance analyzer supported by the housing and operable to measure impedance of the starting material to detect the presence of a contaminant.

### CROSS-REFERENCE TO RELATED APPLICATIONS

This application claims priority to provisional application No. 60/841,774, filed Sep. 1, 2006, and to provisional application 60/876,919, filed Dec. 22, 2006, each of which is incorporated herein by reference in its entirety.

### INTRODUCTION

Avian influenza virus H5N1 was discovered in the late 1990s. Animal cases are reported in more than 46 countries and human infection is reported in 10 countries with 258 people infected and 153 deaths since 2003. Recently, the draft report of the federal government's emergency plan predicts that as many as 200 million Americans could be infected and 200,000 could die within a few months if there were an outbreak of avian influenza in the United States. An unspecified influenza pandemic is projected by the CDC to lead to about 89,000 to 207,000 deaths; 314,000 to 734,000 hospitalizations; 18 to 42 million outpatient visits; and 20 to 47 million additional illnesses in the U.S. The direct economic loss could be up to hundreds of billion dollars. A global influenza pandemic is predicted to cause between 2 million and 7.4 million human deaths worldwide.

In the United States, a recent outbreak of low pathogenic avian influenza (LPAI) in 2001 and 2002 resulted in the depopulation of over 4.5 million chickens and turkeys and is estimated to have cost the poultry industry approximately $125 million. According to the World Bank's report, by mid-2005, more than 140 million birds had died or been destroyed and losses to the poultry industry are estimated to be in excess of US $10 billion.

### SUMMARY

A key in controlling the spread of avian influenza is rapid detection of the disease followed by eradication of infected animals, quarantine within a two-mile radius to prevent movement of people and animals, and vaccination of animals outside the quarantine zone. Currently, techniques used to detect influenza, such as viral culture, RT-PCR and ELISA, are often time consuming, too expensive, or not specific to subtypes of AI viruses. Thus, a simple, rapid, robust and reliable test, suitable for use in the field or at the patient's bedside, is needed.

In one embodiment, an impedance biosensor is provided for detecting a contaminant in a starting material. The biosensor includes an input device, an output device and a microfluidic cell, all of which are supported by a housing. The starting material is engagable with the microfluidic cell and an impedance analyzer which is also supported by the housing and operable to measure impedance of the starting material to detect the presence of a contaminant.

In another embodiment, methods of detecting a contaminant in a starting material are provided. The starting material is contacted with an affinity moiety capable of binding to the contaminant to form a target. The affinity moiety is coupled to a magnetic nanoparticle. The label-free impedance bio sensor described herein is used to detect the target. Detection of the target is indicative of the presence of the contaminant in the starting material.

In yet another embodiment, methods of detecting a virus in a starting material are provided. The starting material is contacted with a red blood cell and the virus is capable of binding the red blood cell to form a complex. The complex is detected with a biosensor. Detection of the complex is indicative of the presence of the virus in the starting material.

In still another embodiment, methods of detecting a contaminant in a starting material are provided. The starting material is contacted with an affinity moiety capable of binding to the contaminant to form a target, wherein the affinity moiety is coupled to a magnetic nanoparticle. The target is detected with an impedance biosensor. Detection of the target is indicative of the presence of the contaminant in the starting material.

In another embodiment, methods of detecting a contaminant in a starting material are provided. The starting material is contacted with an affinity moiety capable of binding to the contaminant to form a target, wherein the affinity moiety is coupled to a magnetic nanoparticle. The target is separated from the starting material and delivered to a microfluidic cell. The microfluidic cell has an interdigitated array microelectrode biosensor disposed therein. The target is detected with the interdigitated array microelectrode biosensor, wherein detection of the target is indicative of the presence of the contaminant in the starting material.

### BRIEF DESCRIPTION OF THE DRAWINGS

FIG. 1 is a front view of a portable biosensor.

FIGS. 2-4 are various views of the biosensor shown in FIG. 1.

FIG. 5 is a view of the interior of the biosensor shown in FIG. 1, shown with a front cover removed.

FIG. 6 is a block diagram of the biosensor shown in FIG. 1.

FIG. 7 is an electrical schematic of the biosensor shown in FIG. 1.

FIG. 8 is a diagram of a cartridge assembly for a biosensor.

FIG. 9 is a top view of a microfluidic flow cell with embedded interdigitated array microelectrodes (IDAM) of the biosensor shown in FIG. 1.

FIG. 10 is a perspective view of a microchannel used in a microfluidic flow cell.

FIG. 11 is a top view of the microchannel shown in FIG. 10.

FIG. 12 is an IDAM chip used in the microfluidic flow cell shown in FIG. 9.

FIGS. 13-15 show a process of fabricating the IDAM chip shown in FIG. 12.

FIG. 16A is a diagram of a label-free biosensor system employing red blood cells and magnetic nanoparticles. FIG. 16B shows a magnetic nanoparticle separation device.

FIG. 17 is a bode plot for the magnitude of impedance vs. frequency for the controls and E. coli O157:H7 with magnetic nanoparticle antibody complexes (MNAC).

FIG. 18 is a graph showing the difference in impedance (represented by the NIC) between E. coli O157:H7 MNAC and control samples with only the MNAC at the indicated frequencies.

FIGS. 19A-19D are a set of graphs showing the impedance of the indicated samples. FIGS. 19A-19B show impedance measurements from a pure culture of bacteria. FIGS. 19C-19D show impedance measurements from E. coli spiked ground beef samples. FIGS. 19A and 19C show the impedance spectrum and FIGS. 19B and 19D show the impedance at 16 kHz.

FIG. 20 is a graph showing the impedance measurements for AI viruses (avian influenza H5N1 virus) based on an immobilized impedance biosensor coated with anti-hemagglutinin (HA) antibodies and pre-incubation with red blood cells.

FIG. 21 is a graph showing the impedance measurements for AI viruses based on an immobilized impedance biosensor coated with anti-HA antibodies and addition of red blood cells after AI binding.

FIG. 22 is a graph showing the impedance measurements for AI viruses based on a label-free impedance biosensor after contacting the AI with magnetic nanoparticles coated with an anti-HA antibody.

FIGS. 23A and 23B are a set of graphs showing impedance measurements made in the presence of avian influenza virus as well as Newcastle and infectious bronchitis viruses. FIG. 23A is a graph showing the impedance measurements for AI viruses in a mixture with Newcastle and infectious bronchitis viruses and red blood cells based on an immobilized impedance biosensor coated with anti-HA antibodies. FIG. 23B is a graph showing the impedance measurements for AI viruses in a mixture with Newcastle and infectious bronchitis viruses based on an immobilized impedance biosensor coated with anti-HA antibodies, wherein red blood cells were added to the biosensor after addition of the antibody.

FIG. 24 is a graph showing the impedance measurements for AI viruses and a mixture of Newcastle and infectious bronchitis viruses based on a label-free impedance biosensor after contacting the AI with magnetic nanoparticles coated with an anti-HA antibody.

FIGS. 25A and 25B are a set of graphs showing the impedance measurements for control tracheal swabs (FIG. 25A) and tracheal swabs spiked with AI (FIG. 25B) after pre-incubation with red blood cells based on an immobilized impedance biosensor coated with anti-HA antibodies.

FIG. 26 is a graph showing the impedance measurements for control tracheal swabs and tracheal swabs spiked with AI based on a label-free impedance biosensor after contacting the AI with magnetic nanoparticles coated with an anti-HA antibody.

FIGS. 27A and 27B are a set of graphs showing the impedance measurements for control cloacal swabs (FIG. 27A) and cloacal swabs spiked with AI (FIG. 27B) after pre-incubation with red blood cells based on an immobilized impedance biosensor coated with anti-HA antibodies.

FIGS. 28A and 28B are a set of graphs showing the impedance measurements for control cloacal swabs and cloacal swabs spiked with ten-fold dilutions of AI after pre-incubation with red blood cells based on an immobilized impedance biosensor coated with anti-HA antibodies. FIG. 28A shows the impedance at a frequency of 10,400 Hz and FIG. 28B shows the impedance spectrum.

FIGS. 29A and 29B are a set of graphs showing the impedance measurements for control cloacal swabs and cloacal swabs spiked with ten-fold dilutions of AI after pre-incubation with red blood cells based on a label-free impedance biosensor after contacting the AI with magnetic nanoparticles coated with an anti-HA antibody. FIG. 29A shows the impedance at a frequency of 4,150 Hz and FIG. 29B shows the impedance spectrum.

FIG. 30 is a bar graph of impedance measurements made with a label-free biosensor system to detect avian influenza virus H5N1 in poultry swab samples.

FIGS. 31A-31E are a set of graphs showing impedance measurements made using a non-commercial antibody against avian influenza virus H5N1.

### DETAILED DESCRIPTION

Before at least one construction of the invention is explained in detail, it is to be understood that the invention is not limited to the details of construction and the arrangements of the components set forth in the following description or illustrated in the drawings. The invention is capable of other constructions and of being practiced or being carried out in various ways.

The ability to detect the presence of small amounts of contaminants, such as bacteria and viruses, in a complex background is of vital importance to biotechnology, medical diagnosis, and prevention of pandemics. Detection and identification of contaminants in infected subjects or within the food or water supply is necessary to protect health and safety. Also, rapid detection of small amounts of contaminants will result in faster clinical diagnosis of disease, and may result in better prognosis. Detection of contaminants is difficult when only a small amount must be detected in a large sample volume or within a complex sample such as a food product or soil. There exists a need in the art for additional methods for detecting and quantifying contaminants that are sensitive, specific and rapid. In addition, there also exists a need for handheld or portable devices for detecting and quantifying contaminants which can be used in the field.

The inventors describe herein methods and systems for detection and quantification of contaminants in a starting material. A microfluidics- and interdigitated array microelectrode-based impedance biosensor is described herein. The described biosensor, which may be implemented in a portable device, allows for highly sensitive, specific, and rapid detection of contaminants and allows for minimum time between samples. Methods are also provided that allow detection of a contaminant in a starting material. The methods include contacting the starting material with an affinity moiety capable of binding to the contaminant to form a target. The affinity moiety can be coupled to a magnetic nanoparticle to form a magnetic target. The magnetic target is then detected, suitably with a biosensor such as the impedance biosensor described herein. In addition, a method is provided for detection of influenza and Newcastle viruses in a starting material by contacting the starting material with a red blood cell to form a complex and detecting the complex using a biosensor, suitably the biosensor described herein.

The biosensor technology is based on one or more concepts, including: (1) the use of magnetic nanoparticles for highly efficient and rapid separation of target virus in a poultry swab sample; (2) a microfluidic biochip with embedded interdigitated array microelectrode for precise delivery and sensitive measurement of the target virus; and (3) formation of complexes of red blood cells and nanoparticles for greater amplification of the impedance signal. Laboratory-scale experiments based on chicken swab samples that were prepared with inactivated avian influenza virus H5N1 and other viruses were completed. The results demonstrate that the biosensor is able to specifically detect avian influenza H5N1 with a limit of detection of at least 100 EID₅₀/ml in a poultry cloacal or tracheal swab sample in less than 30 min. The biosensor will be able to operate in the field, and the cost is estimated at less than $10 per sample. The biosensor described herein introduces the concept of real time detection of avian influenza.

### Impedance Biosensor

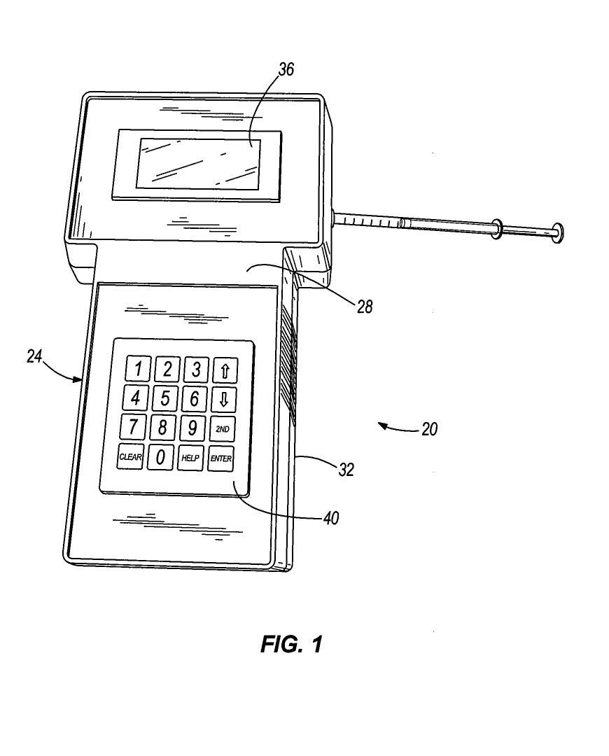

With reference to FIG. 1, an impedance biosensor 20 is illustrated and is operable to detect the presence of and quantify an amount of a contaminant in a solution introduced into the biosensor 20. Contaminants detectable and quantifiable by the biosensor 20 include, for example, bacteria, viruses, eukaryotic cells, polypeptides, or other biological or chemical contaminants. The biosensor 20 is a stand alone device operable to measure impedance values for purposes of detecting and quantifying contaminants in a solution. The biosensor 20 is also easily portable by a user.

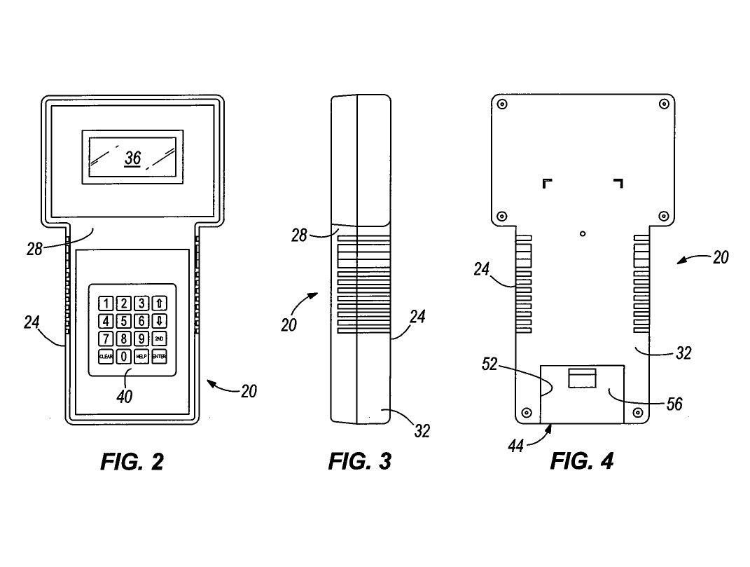

With continued reference to FIG. 1 and additional reference to FIGS. 2-5, the biosensor 20 includes a housing 24 having a front portion 28 and a rear portion 32 removably connectable together by fasteners (not shown). The biosensor 20 also includes a Liquid Crystal Display (“LCD”) 36 and an input device or keypad 40 supported by the front portion 28. The LCD display 36 and keypad 40 provide input and output sources for a user, respectively. A power source receptacle 44 is supported by the rear portion 32 and receives and supports a power source 48, such as, for example a battery or a plurality of batteries, for powering the biosensor 20. In some embodiments, the power source 48 is a pair of AA 1.5 volt batteries and provides DC power. In other embodiments, the power source 48 is an electrical cord and electrical outlet providing AC power. An opening 52 is defined in the rear portion 32 to facilitate insertion and removal of the power source 48 into and from the receptacle 44. A cover 56 is removably connectable to the rear portion 32 in a position over the opening 52 to selectively cover and uncover the receptacle 44.

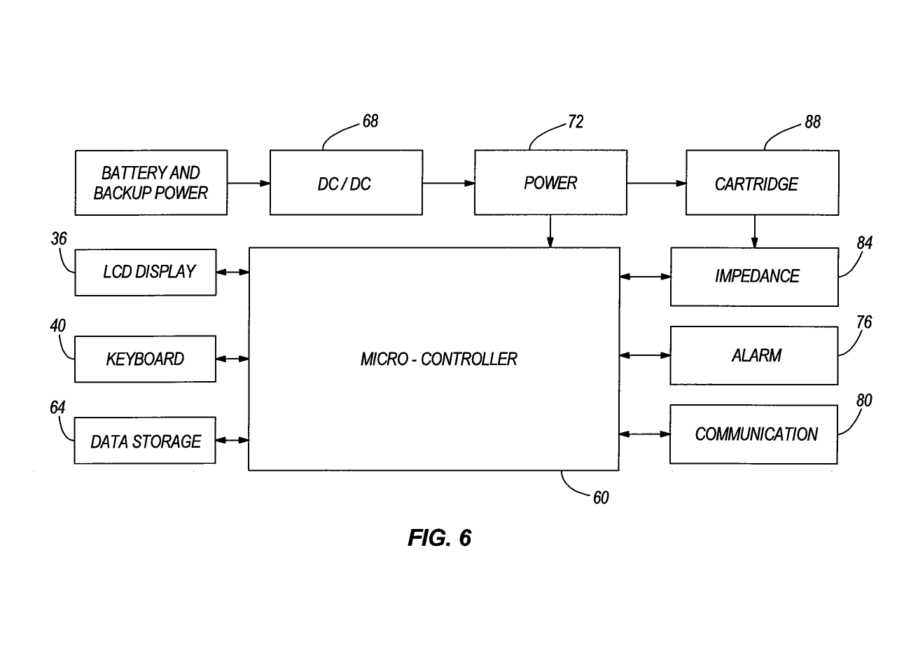

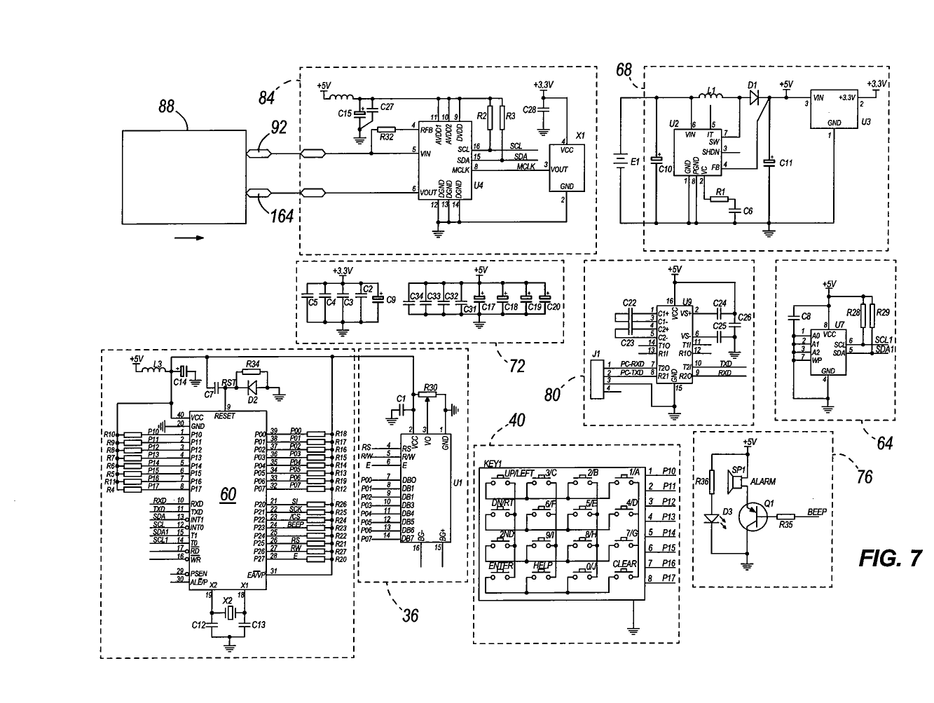

Referring now to FIGS. 6 and 7, the biosensor 20 includes a micro-controller 60, data storage 64, a power converter 68, a power filter 72, an alarm 76, a communication port 80, an impedance detector 84, and a cartridge assembly 88. It should be understood that embodiments of the invention include hardware, software, and electrical components or modules that, for purposes of discussion, may be illustrated and described as if some of the components were implemented solely in hardware and/or software. However, one of ordinary skill in the art, and based on a reading of this detailed description, would recognize that, in at least one embodiment, the electrical and software based aspects of the invention may be implemented in various arrangements of hardware and software. As such, it should be noted that a plurality of hardware and software based devices, as well as a plurality of different structural components may be utilized to implement the invention. Furthermore, and as described in subsequent paragraphs, the specific mechanical configuration illustrated in FIGS. 6 and 7 is intended to exemplify one embodiment of the invention and that other alternative configurations are possible.

As further described below, the micro-controller 60 performs a variety of operations and functions of the biosensor 20, and communicates with various components of the biosensor 20. In one embodiment, the micro-controller 60 is a model no. AT89C55WD microchip IC offered by ATMEL Corporation. Those of ordinary skill in the art will recognize, however, that other components and combinations of components, such as microprocessors, digital signal processors, ASICs, etc. can be used in place of the micro-controller 60. For FIG. 7, the micro-controller 60 is coupled to an oscillator circuit including oscillator X2 and capacitors C12 and C13; and a VCC/reset circuit including inductor L3, capacitors C7 and C14, diode D2, and resistor R34.

The keypad 40 and the LCD 36 provide input and output sources for a user, respectively, although other types of input and output devices may be used. Such input and output devices are conventional in the art and will not be described further herein. For FIG. 7, the LCD 36 is a model no. LCD1604B LCD display offered by OPTREX.

The data storage 64 is supported within the biosensor 20 and facilitates storage of data on-board the biosensor 20. The data storage 64 can consist of a variety of conventional storage means, such as, for example a model no. AT24LC256 memory IC offered by ATMEL Corporation. It should be understood that, while the data storage 64 is shown as a separate memory, in other embodiments the data storage can be combined with other elements of the biosensor 20 (e.g., the micro-controller 60).

The communication port 80 facilitates communication between the biosensor 20 and an external electrical device such as a personal computer, printer, etc. For FIG. 7, the communication port 80 includes capacitors C22, C23, C24, C25, and C26; and device U9, which can be a model no. MAX232 communication IC offered by Maximum.

The impedance detector 84 is operable to sense an impedance of samples introduced into the cartridge assembly 88 and to provide a value having a relationship with the sensed impedance to the microcontroller 60. For FIG. 7, the impedance detector 84 includes inductor L2; capacitors C15, C27 and C28; resistors R2, R3, and R32; oscillator X1; and impedance IC U4, which can be a model no. AD5934 impedance IC offered by Analog Devices, Inc. The impedance detector 84 by itself or in combination with other circuitry (e.g., the microcontroller 60) form an impedance analyzer 85 to, among other things, make a determination based on the sensed impedance. For example, the impedance analyzer 85 can be used to detect the presence of a contaminant. Other determinations for the impedance analyzer 85 will be discussed below.

The power converter 68 receives power from the power source and converts/regulates the power to the proper voltage for the circuitry shown in FIG. 7. For FIG. 7, the power converter includes inductor L1; capacitors C6, C10, and C11; diode D1; voltage IC U2, which can be a model no. LT 1302 voltage IC offered by Linear Technology Corporation; and voltage IC U3, which can be a model no. LX8117-33CDD Voltage IC offered by Microsemi Inc.

The alarm 76 provides a visual and/or audible alarm. For FIG. 7, the alarm 76 includes resistors R35 and R36, LED D3, transistor Q1, and beep SP1.

The power filter 72 filters the VCC voltage and includes capacitors, for FIG. 7, C2, C3, C4, C5, C9, C17, C18, C19, C20, C31, C32, C33, C34.

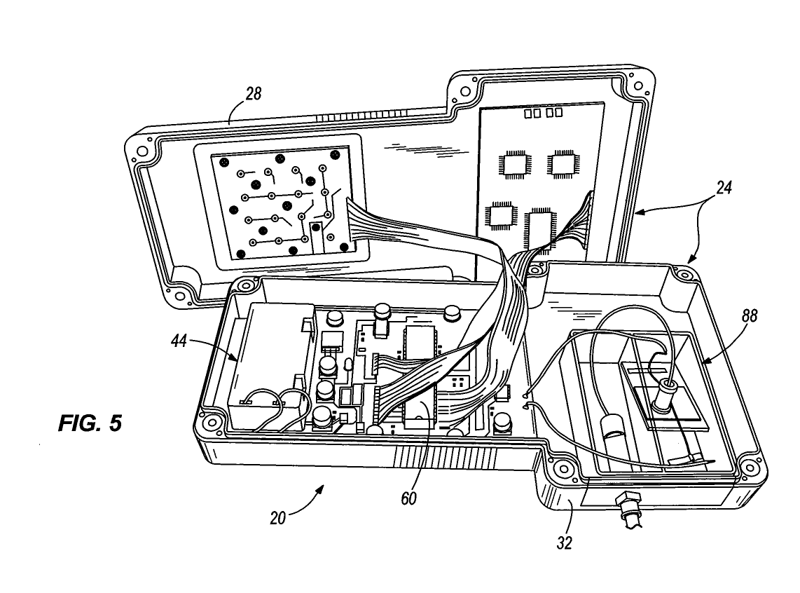

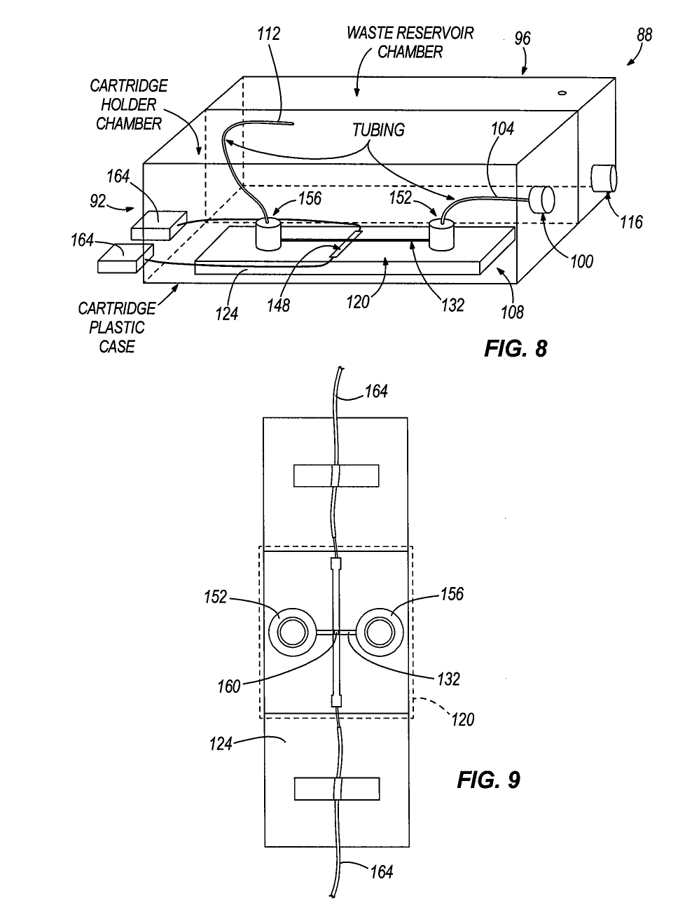

With reference to FIGS. 5 and 8, the cartridge assembly 88 will be described in further detail. The cartridge assembly 88 is positioned in an upper portion of the housing 24 and is divided into a testing portion 92 and a waste reservoir portion 96. A sample is introduced into the testing portion 92 for testing and then, after testing, the sample is dispensed into the waste reservoir portion 96 where the sample or samples accumulate until disposal. The sample is introduced into the biosensor 20 via an injection port 100 accessible from the exterior of the housing 24. Inlet tubing 104 is connected between the injection port 100 and a microfluidic cell 108 (discussed in more detail below) and outlet tubing 112 is connected between the microfluidic cell 108 and the waste reservoir 96. A discharge port 116 is fluidly connected to the waste reservoir 96 to facilitate discharge of the sample(s) from the waste reservoir 96.

### Microfluidic Cell

Microfluidics technology has many advantages, among them high surface to volume ratio, small sample volume, accurate control, and low cost. A microfluidic channel (a cross section of 40 μm depth and 50 μm width) has been designed and fabricated. See Li and Su, 2006, J. Rapid Methods Automation in Microbiol 14: 96-109 (incorporated herein by reference in its entirety). An interdigitated array microelectrode (3 pairs of electrode fingers with 25 μm width) was designed and fabricated into the microfluidic channel for impedance measurement sensitive to a biological target in a sample. Impedance biosensors to detect Salmonella typhimurium and E. coli O157:H7 cells captured onto the surface of an interdigitated array microelectrode modified with specific antibody (Yang et al., 2004, Anal. Chem 76:1107-1113) (incorporated herein by reference in its entirety) or passing through an interdigitated array electrode (Varshney et al., 2007, Biosens Bioelectron, 22(11): 2408-2424) (incorporated herein by reference in its entirety) have been described.

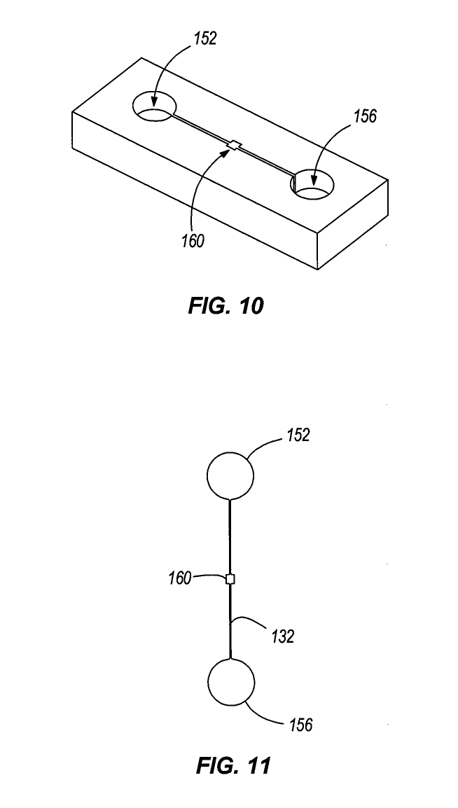

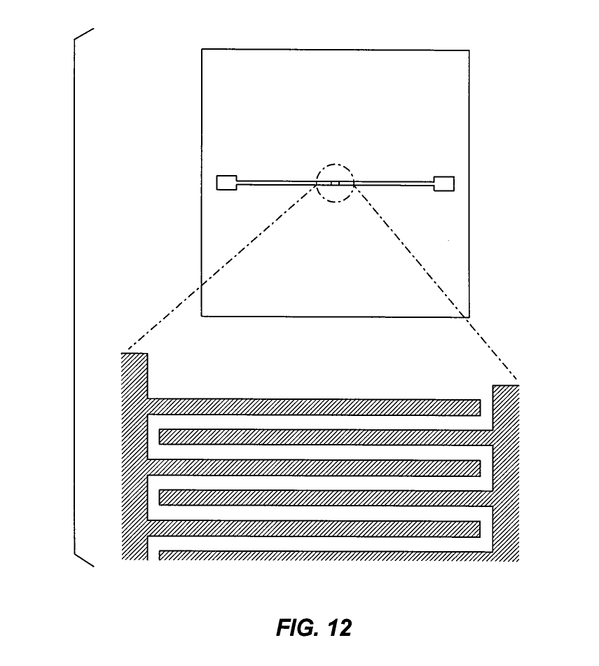

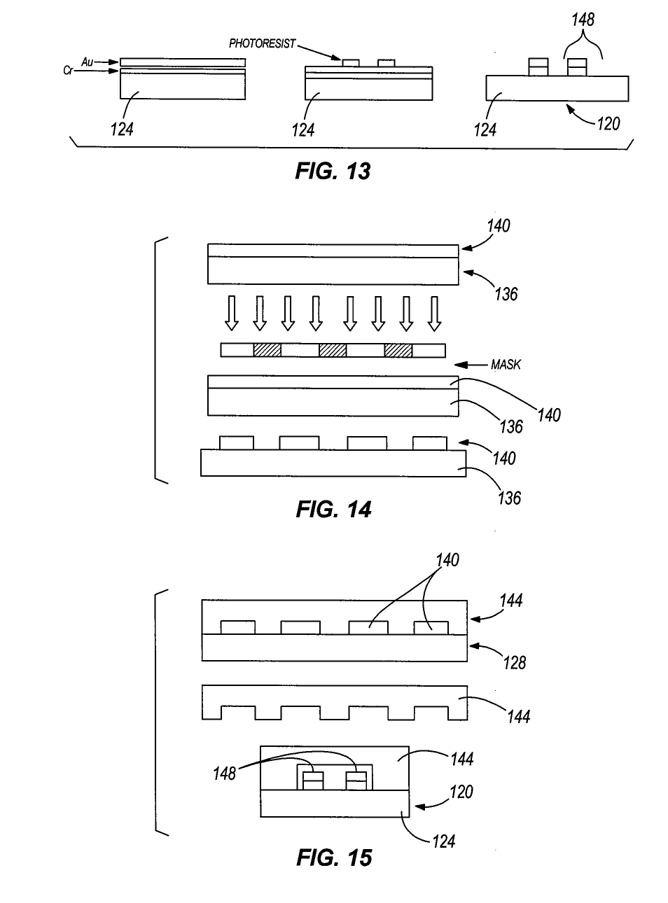

With continued reference to FIGS. 5 and 8 and further reference to FIGS. 9-12, the microfluidic cell 108 is positioned in the testing portion 92 and is fabricated in multiple stages. The fabrication process described herein is for exemplary purposes only. Accordingly, other fabrication processes can be utilized to fabricate a microfluidic cell capable of performing the desired operations and be within the spirit and scope of the present invention.

First, an interdigitated array microelectrode (“IDAM”) chip 120 is fabricated on a glass wafer 124 coated with a gold layer. In this stage, the glass wafer 124 is sputtered with 5000 Å Au with 250 Å Cr as an adhesion layer. The glass wafer 124 is then spin-coated with a 4 μm thick layer of AZ 4330 photoresist. Photolithography is used to pattern the photoresist and then the resist is developed. The Au and Cr on the glass wafer 124 is etched with the patterned photoresist as a masking layer to obtain the electrodes. In some embodiments, the IDAM chip 120 consists of 50 pairs of gold electrode fingers with a finger width of 25 μm and a gap of 25 μm between the fingers.

Second, a mold 128 is prepared for fabricating a microchannel 132. In this stage, a Silicon (Si) wafer 136 is spin-coated with SU-8 140 and soft baked. Then, the Si wafer 136 and SU-8 coating 140 is masked and UV exposed to develop the SU-8 structures 140. The mold 128 is then hard baked.

Third, the microchannel 132 is formed and bonded with the IDAM chip 120. In this stage, polydimethylsiloxane (PDMS) 144 is poured into the mold 128 and partially cured at 60° C. for 1 hour. Then, the PDMS channel 144 is pulled from the mold 128. To bond the PDMS channel 144 on the IDAM chip 120, the PDMS channel 144 is aligned with the microelectrodes 148 and thermally bonded to the chip at 80° C. for 8 hours. The resulting microchannel 132 includes an inlet 152, an outlet 156 and a detection chamber 160 therealong. In some embodiments, the microchannel 132 has a depth of 15 μm and a width of 500 μm, and the detection chamber 160 has a size of 0.5×0.5×0.02 mm and a volume of 5 nl. The microfluidic cell 108 also includes electrical connectors 164 connected to the IDAM to provide power thereto and to communicate information between the IDAM and the micro-controller 60.

### Impedance Biosensor Operation

Now that the biosensor 20 has been described, the purpose and exemplary operation of the biosensor 20 will be described. The biosensor 20 is operable to detect and quantify the presence of a contaminant within a sample introduced therein by utilizing impedance measurements.

The following operation of the biosensor 20 is one manner of detecting and quantifying the presence of contaminant in a sample and is not meant to be limiting. Accordingly, other manners of detecting and quantifying contaminants, for example using other biosensors, are possible and are within the spirit and scope of the present invention.

In some embodiments, the interdigitated array microelectrode operates as follows. The flow of current across the interdigitated array microelectrode, specifically across the space between the fingers, is impeded by the presence of targets in the channels. The impedance is in proportion to the amount of target that is present and is a function of the frequency of the voltage signal that is applied.

After the sample is contacted with an affinity moiety, and affinity moiety-contaminant “targets” are formed, the targets are separated from the rest of the sample and are subsequently quantified using the biosensor 20. If the affinity moieties are coupled to magnetic nanoparticles, then a magnetic separator can be used to remove the beads-plus-target complexes (see below for a further discussion of magnetic nanoparticles).

An aspect of the present system is the mechanism for contacting the targets with the interdigitated array microelectrode, specifically the use of a continuous flow system. By using a continuous flow system, multiple samples can be run through the biosensor 20 in series, with minimal downtime between samples.

Another aspect of the present system is the fact that the affinity moiety does not have to be integrated into the interdigitated array microelectrode (termed a “label-free” biosensor), but rather in some variations can be made part of the flow-through material. Because of this feature, the biosensor 20 can be reused with numerous different affinity moieties and thus can be used to detect a variety of different contaminants, without having to change microelectrodes. In other systems, by contrast, the affinity moiety (typically an antibody) is coupled to the microelectrode, with the result that the particular microelectrode can only be used to detect one contaminant. Furthermore, samples can be analyzed more rapidly with a label-free system because there is no residual material to be stripped or removed from the biosensor between readings as is the case for an “immobilized” biosensor, i.e. a biosensor having an affinity moiety attached thereto.

After the targets are introduced into the biosensor 20, oscillating voltage signals can be applied at a range of frequencies or at a single frequency, and the resulting impedances are recorded. In the absence of a positive reaction between contaminants in the sample and the affinity moiety, the affinity moieties will not form target complexes and thus will have a measurably different profile on the impedance spectrum. Conversely, when there is a positive reaction, the target complexes of affinity moieties plus contaminant that are formed will cause a measurable increase in impedance in certain portions of the frequency spectrum, producing a distinctive signature.

In a typical measurement, a sample solution is introduced into the injection port 100 and travels to the microfluidic cell 108 via the inlet tubing 104 and inlet 152 of the microchannel 132 (FIG. 8). The microfluidic cell 108, including the fabricated IDAM chip 120 and microchannel 132, is used to collect contaminant in an active layer above the IDAM and minimizes interfering effects of particulates in the sample while impedance is being measured. The active layer is a few microns above the surface of the IDAM and is where the electric field has maximum strength. The magnitude and phase angle of impedance can be measured in a frequency range from 10 Hz to 1 MHz in the presence of 0.1 M mannitol solution. Alternatively, the magnitude and phase angle can be measured at different frequencies in the presence of other solutions and concentrations. After the sample passes through the microchannel 132, the sample exits the microfluidic cell 108 via the outlet 156 of the microchannel 132 and the outlet tubing 112. Next, the sample travels to the waste reservoir 96 and is eventually dispensed from the biosensor 20 through the discharge port 116.

Impedance measurements of the solution can be conducted by the impedance biosensor system described above or by a conventional impedance detector or analyzer, such as, for example an IM-6 impedance analyzer manufactured by BAS of West Lafayette, Ind. and with conventional software, such as, for example IM-6/THALES software. For all impedance measurements, a sine-modulated AC potential of 100 mV was applied across the IDAM and the magnitude and phase angle of impedance is measured for a frequency range from 10 Hz to 1 MHz. Using preformed magnetic nanoparticle antibody complexes (MNAC), the total detection time from sampling to measurement was 35 minutes. One pole of the IDAM chip is connected to test and sense probes, and the other pole is connected to reference and counter electrodes of the impedance analyzer.

In order to determine a frequency for the maximum difference in impedance measurements between the sample solution with attached MNAC and the control, a curve was drawn between normalized impedance change (NIC) and frequency. The value of NIC was given by the following formula (1):

$$\frac{{Z}_{sample} - {Z}_{control}}{{Z}_{control}} \times 100 \qquad ( 1 )$$

where, Z_{control} is the magnitude of impedance for a control, and Zₛₐₘₚₗₑ is the magnitude of impedance for a sample solution.

The methods described herein may be used to detect the presence of contaminants, including influenza virus, in a wide variety of starting materials with various levels of complexity in terms of antigenic diversity, density and volume. In addition to the starting materials used in the examples below, it is reasonable to expect that contaminants may be detected in a wide variety of food products, animals, and environmental and clinical samples, and may include liquid, solid or materials containing a mixture of liquids and solids. The starting materials may include vegetables, fruits, ground meats, beef, poultry, sea food, dairy, water, air, soil, blood, urine, feces; swabs from the surface of the skin, organs, trachea, or cloaca; or tissue samples. Food samples may be raw or ready-to-eat. For example, a poultry product may include a carcass, wash water from a carcass, a deboned bird, ground poultry meats, or poultry patties. The methods are also suitable for food or environmental inspection or clinical diagnosis or monitoring. For example, the methods may be used to monitor food during processing, storage, distribution or even once in the market. A solid or semi-solid starting material may be subject to homogenization prior to use in the methods.

As described in the Examples below, many types of contaminants may be detected or quantified using the methods described herein. In the Examples, Escherichia coli O157:H7 and avian influenza H5N1 were detected. In addition to these contaminants, it is reasonable to expect that one of skill in the art may use the methods with a wide variety of potential contaminants including, but not limited to, bacteria such as Listeria monocytogenes, Campylobacter jejuni, Pseudomonas mirabilis, Salmonella species and Enterococcus species; eukaryotic cells; polypeptides, including prions, toxins and blood or urine proteins; viruses such as influenza; or other chemical contaminants such as pesticides or herbicides. Significantly, both live and dead cells may be detected by the methods described herein. Starting materials, however, may be pre-treated to kill any live contaminants that may pose a health risk to a technician performing the method described herein.

### Magnetic Nanoparticles

Nanoparticles have previously been described. See Varshney et al., J Food Protection 68:1804-1811 (2005); Fritzsche and Taton, Nanotechnol. 14:R63-R73 (2003); Tan et al., Med. Res. Rev. 24:621-638 (2004); Zhao et al., PNAS 101:15027-15032 (2004); U.S. Pat. No. 6,623,982; and U.S. Pat. No. 6,645,731 all of which are incorporated herein by reference in their entireties. Nanoparticles range in size from 1-300 nm in diameter, suitably from 50-150 nm in diameter. Nanoparticles may have a magnetic core that may include various metals and, like microbeads, the magnetic core may be paramagnetic or superparamagnetic. Nanoparticles suitable for use in the methods described herein include those commercially available from Molecular Probes, Inc. (Eugene, Oreg.).

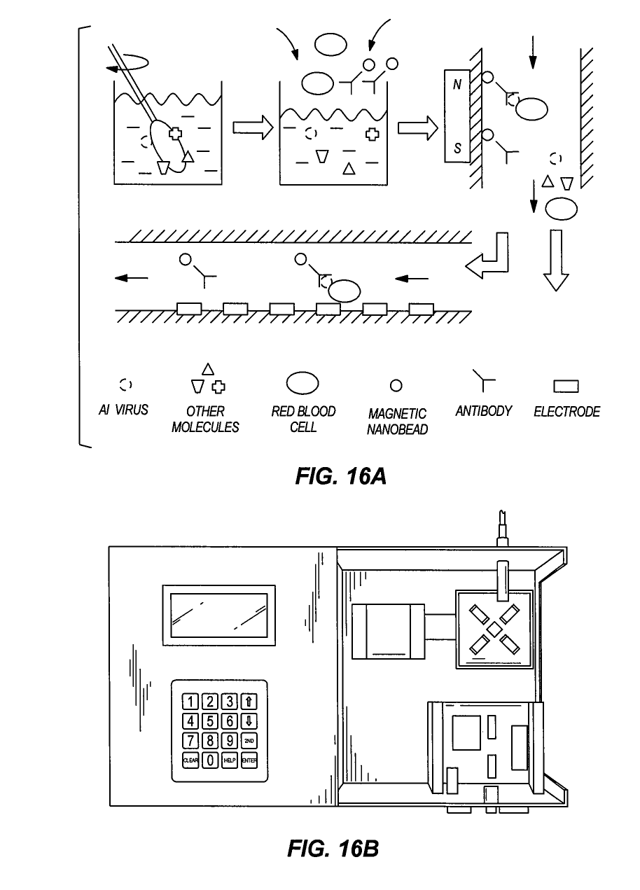

Nanoparticles from 50-150 nm exhibit properties of ferrofluids and remain stable colloids. They can be collected in a magnetic monolayer which allows washing away of unwanted background. Compared to other separation techniques such as centrifugation, filtration, and use of microbeads, magnetic nanoparticles provide higher capture efficiency of avian influenza virus in a poultry sample, which in turn enhances the sensitivity of the biosensor. Once the nanoparticles are contacted with the starting material and the magnetic target is formed, the magnetic target may be separated from the starting material in a variety of ways. The target may be separated by filtration or centrifugation, or by generation of a magnetic field. Magnetic separation devices suitable for use in the methods include the Magnetic Particle Concentrator (MPC) from Dynal, Inc. (Lake Success, N.Y.) and the separation system described in U.S. patent application Ser. No. 11/328,808, entitled “Separation System and Efficient Capture of Contaminants Using Magnetic Nanoparticles,” incorporated herein by reference in its entirety (FIG. 16B).

The nanoparticles may be directly or indirectly coupled to affinity moieties having affinity for the contaminant. Nanoparticles are commercially available already prepared for coupling to affinity moieties by a variety of chemical reactions, but may also be prepared by the end-user. Nanoparticles may be coupled to affinity moieties by a variety of methods including, but not limited to, pre-conjugation to streptavidin, avidin, Protein G or Protein A; using commercially available kits, nanoparticles can be directly bound to antibodies via a covalent linkage; nanoparticles with functional carboxy or amino groups exposed on their surface for use in coupling a variety of polypeptides; or nanoparticles linked to a polypeptide (a linker) capable of binding either the Fc region of an antibody, such as an Fc receptor or anti-Fc antibody, or a non-Fc region of the antibody. Polypeptides may be biotinylated by methods well known to those of skill in the art and the biotin may form a bridging complex linking a nanoparticle to an affinity moiety by binding to streptavidin or avidin. It is reasonable to expect that one of skill in the art may utilize a variety of different chemistries to couple the antibody to the nanoparticle either directly or indirectly.

The magnetic nanoparticles are able to bind to the contaminant by virtue of their coupling to an affinity moiety. Affinity moieties are suitably polypeptides that have affinity for the contaminant and will bind to the contaminant when brought into proximity with it. Affinity moieties include antibodies specific for the contaminant, ligands capable of binding a receptor on the contaminant, and receptors that bind to the contaminant. In addition to those affinity moieties exemplified below as useful in the methods described herein, it is reasonably expected that other affinity moieties with affinity for various contaminants will be suitable. Suitable antibodies may be identified using an antibody source guide, for example Linscott's Directory of Immunological and Biological Reagents or the MSRS Catalog of Primary Antibodies. Methods for generating monoclonal and polyclonal antibodies are known to those of skill in the art.

The steps of the method may be completed in several different orders. For example, the antibody may be coupled to the magnetic nanoparticle prior to contact with the starting material, or it may be added after the contaminant-antibody target is formed. Alternatively, the antibody and the magnetic nanoparticle may be added to the starting material simultaneously.

Biosensors may be utilized to detect and quantify the contaminant in the starting material. Suitable biosensors include but are not limited to, impedance biosensors, quartz crystal microbalance biosensors and viscoelastic biosensors such as that described in U.S. patent application Ser. No. 11/329,009, entitled “Method for Detecting an Unknown Contaminant Concentration in a Substance,” incorporated herein by reference in its entirety. Suitably, the biosensor described herein is utilized to detect and quantify the contaminant in the starting material.

Influenza and Newcastle viruses are capable of hemagglutinating red blood cells to form virus-red blood cell complexes. The inventors describe a method of detecting a virus in a starting material by contacting the starting material with a red blood cell. The virus in the starting material is capable of binding to the red blood cells and the complex of red blood cell-virus can then be detected using a biosensor. Suitably an impedance biosensor is used to detect the virus-red blood cell complex, more suitably the impedance biosensor described herein is utilized. The virus-red blood cell complex can be further contacted with an antibody to provide specificity to the biosensor. For use in the biosensor described herein, the antibody is coupled to a magnetic nanoparticle. As described above, the steps in the methods may be performed in a variety of orders. For example, the virus may be bound to the affinity moiety-nanoparticle complex and then incubated with red blood cells or vice versa. In an immobilized antibody-based impedance biosensor, the virus may be complexed with red blood cells prior to addition to the biosensor, or after the virus is captured in the biosensor by the affinity moiety.

In one particular embodiment illustrated in FIG. 16A, magnetic nanoparticles are coated with virus-specific antibodies and used to separate target virus from a poultry swab sample. Red blood cells, as biolabels, can be mixed with the captured target virus to form the nanoparticle-virus-red blood cell complex. A microfluidic biochip is designed and fabricated as a flow-through device to deliver the complex to an embedded interdigitated array microelectrode for impedance measurement. The change in impedance of the nanoparticle-virus-red blood cell complex is correlated to the concentration of avian influenza virus H5N1 in the original sample.

The following examples are meant only to be illustrative and are not intended as limitations on the claims.

### EXAMPLES

### Example 1

### Materials and Methods

The following materials and methods were used throughout the Examples, unless otherwise indicated.

### Culture and Plating of Bacteria

Frozen stock of E. coli O157:H7 (ATCC 43888) was maintained in brain heart infusion broth (BHI, Remel Inc., Lenexa, Kans.) at −70° C. The culture was harvested in BHI maintained at 37° C. for 18 to 22 h. For enumeration, pure cultures were serially diluted in 0.01 M, pH 7.4 phosphate buffered saline (PBS) and surface plated on sorbitol MacConkey (SMAC) agar (Remel Inc., Lenexa, Kans.), which was incubated at 37° C. for 20 to 22 h.

### Avian Influenza Virus

The avian influenza virus (H5N1) is produced by growth in chicken embryos and collection of allantoic fluid and killed by the National Veterinary Services Laboratory, Ames Iowa. The stock solution of virus contains approximately 1×10⁷ egg-infectious doses (“EID”) per ml.

### Chemicals and Reagents

PBS (0.01 M, pH 7.4) was obtained from Sigma-Aldrich (St. Louis, Mo.). Bovine serum albumin (BSA; EM Science, Gibbstown, N.J.), 1.0% (wt vol⁻¹) was prepared in PBS as a blocking buffer (PBS BSA). Protein A (from Staphylococcus aureus Cowan strain cell walls) was obtained from Sigma-Aldrich (St. Louis, Mo.). One-tenth molar (0.1 M) solution of mannitol (Sigma-Aldrich, St. Louis, Mo.) in deionized water was used for washing and resuspension of bacteria and viruses and was used for washing electrodes. All solutions were prepared with deionized water from Millipore (Milli-Q, 18.2 MΩ·cm, Bedford, Mass.).

### Nanoparticles and Antibodies

Magnetic nanoparticles (average diameter 145 nm, 0.5 mg Fe ml⁻¹) conjugated with streptavidin were obtained from Molecular Probes Inc. (Eugene, Oreg.). Magnetic nanoparticles have more than 85% of oxide as Fe₃O₄, approximately 80% wt wt⁻¹ of magnetite, and approximately 4×10¹¹ particles mg⁻¹ Fe.

Affinity-purified polyclonal goat antibodies against E. coli (specific for O and K antigens) conjugated with biotin were obtained from Biodesign International (Saco, Me.). The concentration of stock solution of biotin-labeled antibodies was 4 to 5 mg ml⁻¹. A 1:10 dilution of the antibodies was prepared in PBS (0.01 M, pH 7.4) before use.

Rabbit antibody to influenza A H5N1 Hemagglutinin (HA) (hereinafter referred to as “anti-HA antibody”) was purchased from Biodesign International (Saco, Me.). This affinity-purified antibody was raised in rabbit against a synthetic peptide (Genbank accession no. AAT76166) corresponding to 14 amino acids in the middle region of the hemagglutinin protein.

### Immunomagnetic Separation and Concentration of Bacteria

Magnetic nanoparticle antibody complexes (MNAC) were prepared in 1.7 ml sterile polypropylene centrifuge tubes. Biotin-labeled polyclonal goat anti-E. coli antibodies (7.5 μl) were continuously mixed with streptavidin-coated magnetic nanoparticles (15 μl) in 250 μl PBS BSA at 7 rpm on a variable speed rotator (ATR, Laurel, Md.) for 35 min at room temperature. Following antibody immobilization, MNAC were mixed with 150 μl of biotin solution (in PBS BSA) for 15 min to block unbound streptavidin present on the surface of magnetic nanoparticles. Excess biotin was washed out with PBS-BSA, and MNAC were resuspended in 450 μl of PBS BSA.

Serial dilutions of pure culture of E. coli O157:H7 from 8.4×10² to 8.4×10⁸ CFU ml⁻¹ were prepared in PBS (0.01 M, pH 7.4) buffer. A 50 μl aliquot of pure culture was mixed with 450 μl of MNAC for an immunoreaction time of 15 min. Following the immunoreaction, nanoparticle-bacteria complexes were washed three times with 0.1 M mannitol solution with an intermittent magnetic separation, and were concentrated in 100 μl of mannitol solution. Finally E. coli O157:H7 cells attached to MNAC suspended in mannitol solution was injected into the flow cell with the help of a syringe pump at a flow rate of 10 μl/min. Similar procedures were followed for viral detection.

### Biotinylation of AI H5N1 Antibodies

Biotinylation of Avian Influenza (AI) H5N1 antibody was performed with EZ-Link Sulfo-NHS Biotinylation Kit (obtained from PIERCE (Rockford, Ill.)) according to the supplied instructions. Briefly, 100 μl anti-HA (1 mg/ml) was mixed with 3 μl Sulfo-NHS-Biotin solution (10 mM) into 200 μl PBS (10 mM, pH 7.4) and incubated at room temperature for 60 min. Then, excess biotin was removed by using Slide-A-Lyzer Dialysis Cassettes. The level of biotin incorporation was measured to be 4 to 5 mole biotin per mole antibody by using HABA ([(2-(4′-Hydroxyazobenzene) Benzoic Acid]) assay.

Conjugation of Magnetic Nanoparticles with Anti-HA Antibodies

Biotin-labeled H5N1 antibodies (anti-HA antibodies; 50 μl) were continuously mixed with streptavidin-coated magnetic nanoparticles (20 μl) in 50 μl PBS at 15 rpm on a variable speed rotator (ATR, Laurel, Md.) for 40 min at room temperature. After antibody immobilization, magnetic nanoparticles were mixed with 100 μl biotin solution (1 mg/ml in PBS) for 20 min to block excess streptavidin present on the surface of magnetic nanoparticles. Excess biotin was washed out by a magnetic separation.

### Magnetic Nanoparticle-Antibody Conjugates for Separation and Concentration of AI Viruses

Cloacal swab samples from birds mixed with a 10-fold serial dilution (10⁻², 10⁻³, 10⁻⁴, and 10⁻⁵) of inactivated AI H5N1 virus (original titer was 1×10⁷±one log EID₅₀/ml) were suspended in 500 μl buffer (Isotonic Dextrose with Heparin: 60 grams dextrose per liter distilled water), and a cloacal swab without viruses was used as a control. The 500 μl of swab sample was then mixed with antibody-coated nanoparticles (coated with the anti-HA antibody) for an immunoreaction time of 30 min. After immunoreaction, nanoparticle-virus complexes were washed with 300 μl 0.1 M mannitol solution with an intermittent magnetic separation, and then were resuspended in 150 μl of 0.1 M mannitol solution for impedance measurement. Unless otherwise indicated, swab samples were prepared using a solution containing a virus titer of 10̂5 EID₅₀/ml.

### Swab Sample Preparation

Cloacal and tracheal swab samples from birds were mixed with 10-fold serial dilutions (10⁻², 10⁻³, 10⁻⁴, and 10⁻⁵) of inactivated AI H5N1 virus (original titer was 1×10⁷±one log EID₅₀/ml). The swabs were suspended in 2 ml buffer (Isotonic Dextrose), and cloacal or tracheal swab without viruses was used as a control.

### Immobilization Procedure

For some experiments, it was desired to pre-coat the microelectrode with antibody to H5N1 in order to immobilize viruses on the microelectrode for subsequent analysis, using the following procedures. Firstly, Protein A (1 mg/ml in 10 mM PBS, pH 7.4) was injected into the biosensor and incubated at room temperature for ˜1.5-2 hours. Then, the microelectrode was further modified with H5N1 antibody (incubation for 2.5 hours at room temperature). After blocking with BSA (1% in 10 mM PBS, pH 7.4 and incubate at room temperature for 30 minutes), the chip was ready for a swab sample test. After each immobilization step, the microelectrode was washed with 0.1 M mannitol solution, and impedance measurements were performed.

### Impedance Measurement

Impedance measurement was performed using an IM-6 impedance analyzer (BAS, West Lafayette, Ind.) with IM-6/THALES software. For all impedance measurements, a sine-modulated AC potential of 100 mV was applied across the IDAM and the magnitude and phase angle of impedance were measured for a frequency range from 10 Hz to 1 MHz. Using preformed magnetic nanoparticle antibody complexes (MNAC), the total detection time from sampling to measurement was 35 min (15, 10, and 10 min for immunoreaction, washing, and measurement, respectively).

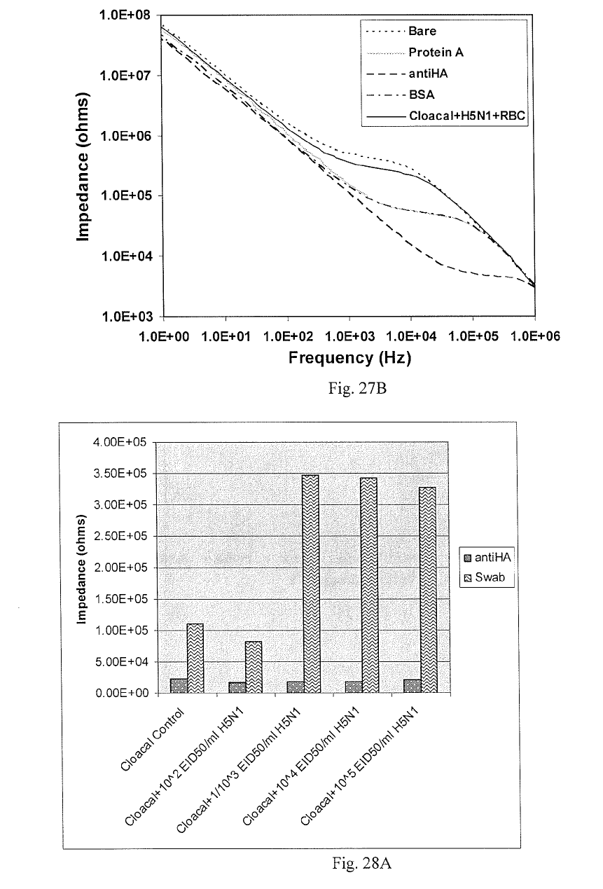

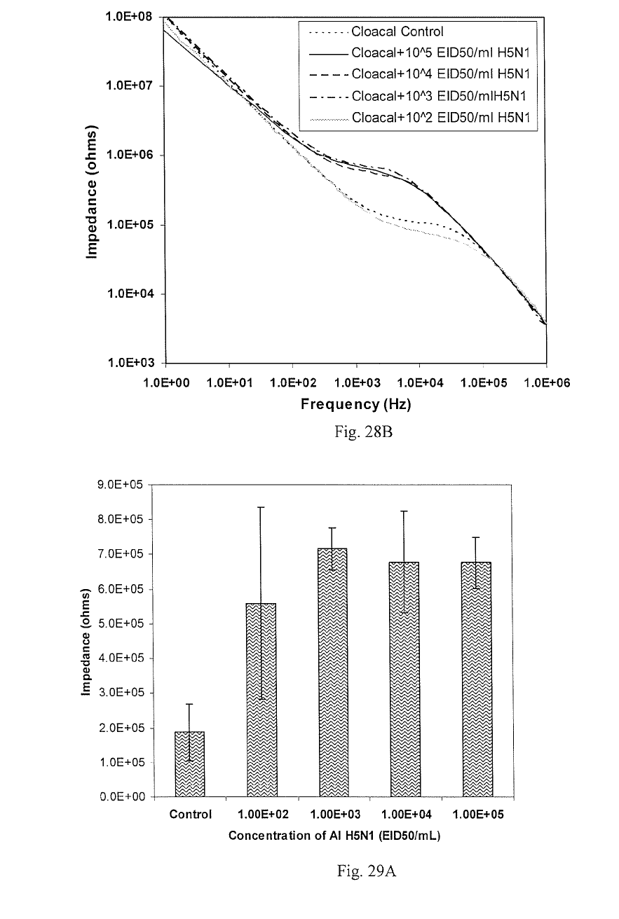

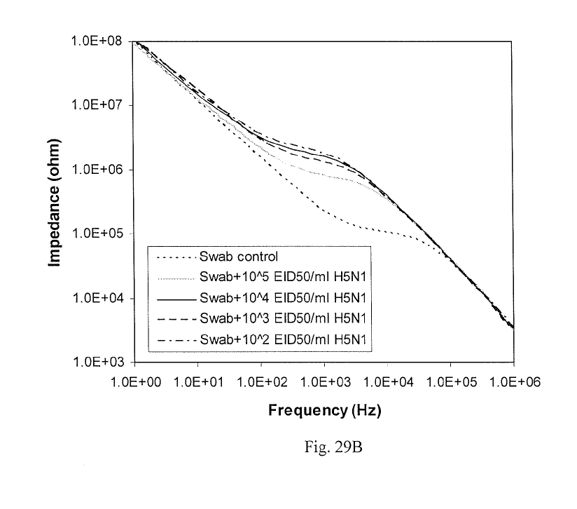

In some embodiments (including the experiments corresponding to the data shown in FIGS. 28A and 29A) a portable, hand-held unit, such as the impedance biosensor 20 that is described above, was used to collect data. In particular embodiments, the hand-held unit collects data at a single frequency rather than a continuous range of frequencies. The single frequency is suitably between 1 Hz and 1 MHz, more suitably between 100 Hz and 10 kHz, and still more suitably between 1 kHz and 10 kHz.

One pole of the IDAM chip was connected to test and sense probes, and the other pole was connected to reference and counter electrodes of the impedance analyzer. At the end of each test, the flow cell was washed with 0.1 M sodium hydroxide for 1 h, deionized water for 30 min, 0.1 M hydrochloric acid for 1 h, and a final rinse with deionized water for 1 h. The flow rate used during washing was 10 μl/min. Mannitol solution with MNAC but no E. coli O157:H7 was used as a control for all tests.

In order to determine a frequency for the maximum difference in impedance measurements between the sample of bacterial cells with attached MNAC and the control, a curve was drawn between normalized impedance change (NIC) and frequency. The value of NIC was given by following formula:

$$N I C = \frac{{Z}_{sample} - {Z}_{control}}{{Z}_{control}} \times 100 \qquad ( 1 )$$

where Z_{control} is the magnitude of impedance for a control and Zₛₐₘₚₗₑ is the magnitude of impedance for a sample containing E. coli O157:H7. Equivalent calculations were performed for other samples including virus samples.

### Preparation of Ground Beef Samples

Ground beef was purchased from a local supermarket. A sample of 25 g of ground beef was homogenized with 225 ml of 0.1% buffered peptone water in a Whirl-pak plastic bag using a laboratory Stomacher 400 (Seward, Norfolk, UK) for 2 min. After stomaching, the sample was centrifuged two times at 250×g for 15 min in order to separate large size particles present in the ground beef stomaching water. The supernatant of the food samples was inoculated with 10-fold dilutions of E. coli O157:H7 cultures ranging from 7.9×10² to 7.9×10⁸ CFU ml⁻¹.

### Example 2

### Impedance Measurement of E. coli O157:H7

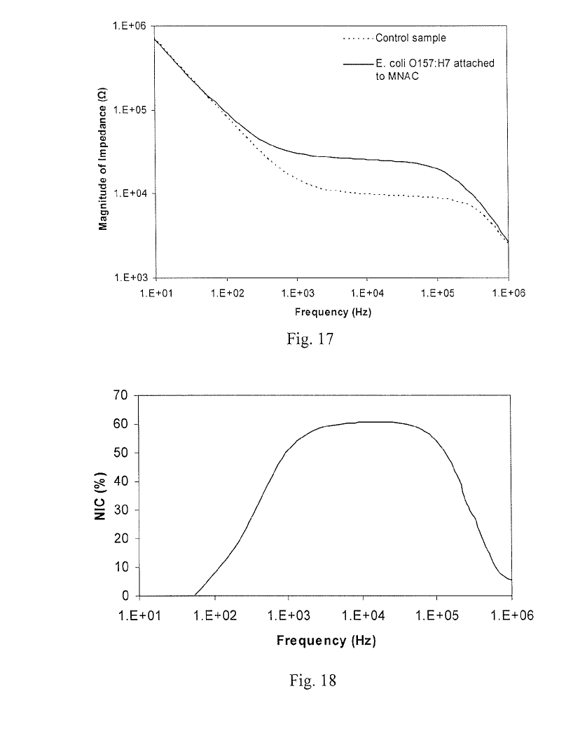

FIG. 17 shows a bode plot corresponding to impedances measured in the presence of 8.4×10⁷ CFU ml⁻¹ of E. coli O157:H7 with attached MNAC as well as a control (with MNAC only and no bacteria). Samples were injected into a microfluidic flow cell containing a label-free biosensor, which was used to measure impedances. Impedance measurements were made in 0.1 M mannitol. The impedance difference (represented by NIC in FIG. 18) showed an increasing trend (from 0.22 to 61%) in the region of 53 Hz to 16 kHz followed by the decreasing trend (61 to 6%) in the region of 16 kHz to 1 MHz. The maximum difference in impedance between measurements was in the bulk medium resistance region (1 kHz to 50 kHz) and peaked at 16 kHz as shown in FIG. 18.

### Example 3

### Detection Limit of the Impedance Biosensor for the Detection of E. coli O157:H7 in Pure Culture and Ground Beef Samples

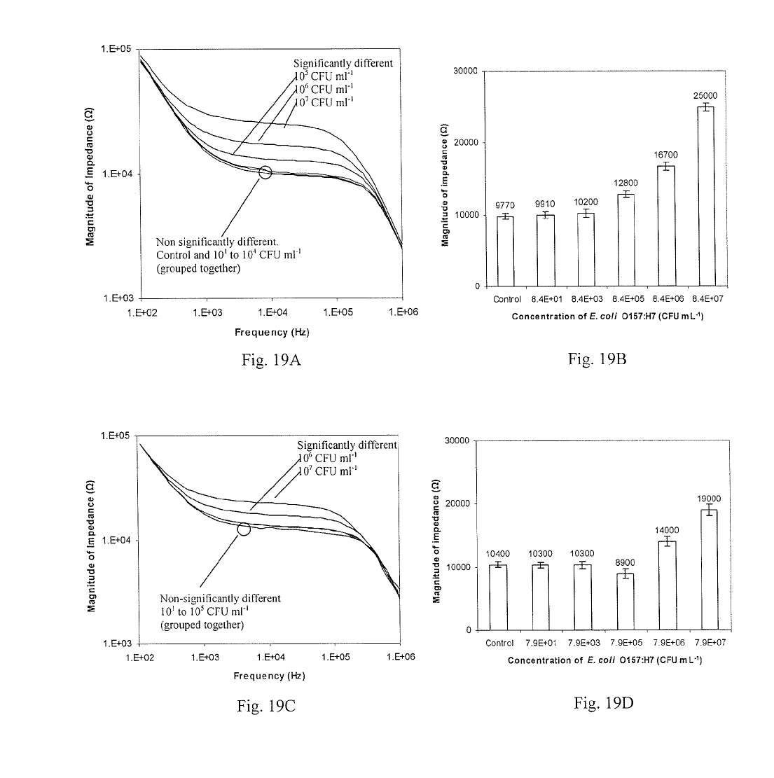

FIGS. 19A, 19B, 19C, and 19D show the measured impedance (measured using a label-free biosensor) for all concentrations of E. coli O157:H7 from 10¹ to 10⁷ CFU ml⁻¹ that are present in a pure culture and in ground beef samples. The impedance caused by bacteria was found to increase linearly with the number of cells in the sample. The polarization and insulating effect of bacteria on the biosensor surface only begins to change impedance at a concentration of 10⁵ CFU ml⁻¹ or higher for pure cultures and 10⁶ CFU ml⁻¹ or higher for samples mixed with ground beef. FIGS. 19B and 19D show a snapshot of the impedance at a frequency of 16 kHz and demonstrate that there were detectable, statistically-significant differences in the impedance between the control sample and bacterial concentrations from 10⁵ to 10⁷ CFU ml⁻¹ in pure culture and 10⁶ to 10⁷ CFU ml⁻¹ in ground beef samples.

### Example 4

### Detection of Avian Influenza Virus with an Impedance Biosensor with Antibodies Immobilized Thereon

Poultry swab samples were prepared by dipping the swab, which may contain AI viruses (50-100 nm diameter), non-AI viruses, and other molecules, into Isotonic Dextrose buffer solution. Then, 1 ml of a 0.5% suspension of red blood cells was added into the swab sample solution and mixed for 1-5 min to form red cell-virus complexes. Influenza viruses, as well as Newcastle viruses, are capable of hemagglutinating red blood cells to form virus-red blood cell complexes.

The swab sample with the complexes was sent to a microfluidic channel (40 μm in depth, 100 μm in width, and 10 mm in length). An interdigitated array microelectrode (15 μm for both electrode finger width and space between electrode fingers) was embedded in the microfluidic channel, and coated with AI virus polyclonal antibodies which were bound by Protein A, producing what is referred to herein as an “immobilized” biosensor. When passed through the microfluidic channel, the AI virus-red cell complexes were specifically captured by the immobilized AI virus antibodies (anti-HA antibodies), resulting in changes in impedance. A 0.1 M mannitol wash solution was applied to wash away loosely attached non-AI molecules before the impedance was measured.

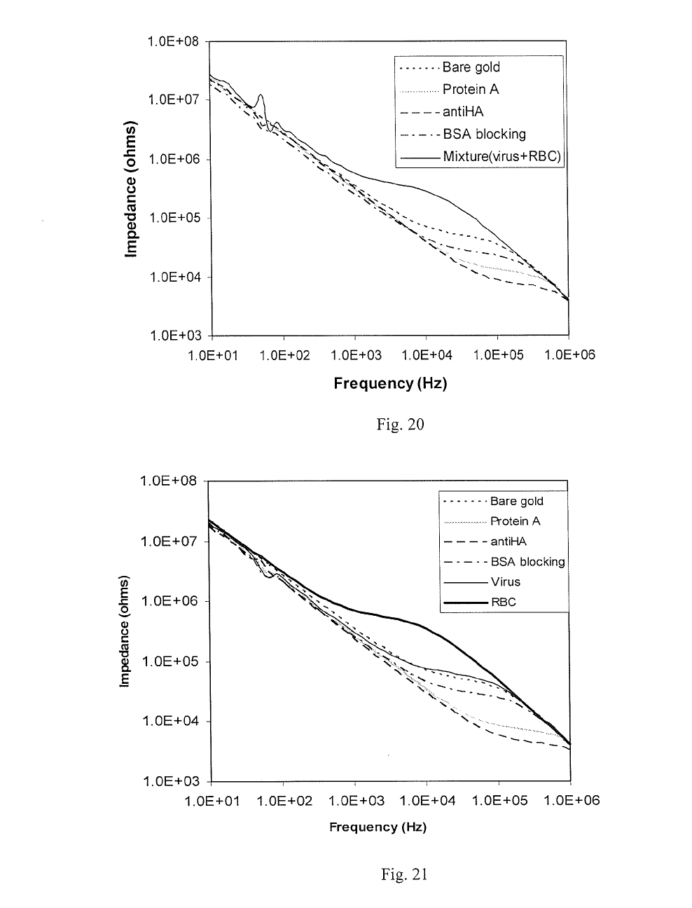

Impedances were measured using an impedance analyzer at frequencies between 1 Hz and 1 MHz. The results are depicted in FIG. 20 and demonstrate that the virus-red cell complexes caused a significant increase in impedance. FIG. 21 demonstrates that the AI virus alone did not result in a significant change in impedance, but addition of red blood cells to the biosensor following addition of the virus resulted in a significant change in impedance.

### Example 5

### Detection of Avian Influenza Virus with an Impedance Biosensor Using Magnetic Nanoparticle Antibody Complexes

Magnetic nanoparticles (MNACs) were coupled to polyclonal antibodies specific for AI viruses (anti-HA antibodies). The nanoparticle-antibody complexes were used in the automatic magnetic sampler to separate and concentrate target viruses from a poultry swab sample. Red blood cells, which are employed as biolabels, were mixed with the captured target viruses to form the affinity moiety-coated nanoparticle-virus-red cell complex as described. The complexes were then delivered to a label-free, flow-through impedance biosensor, as described above, for impedance measurements. Changes in impedance of the affinity moiety-coated bead-virus-red cell complex were measured.

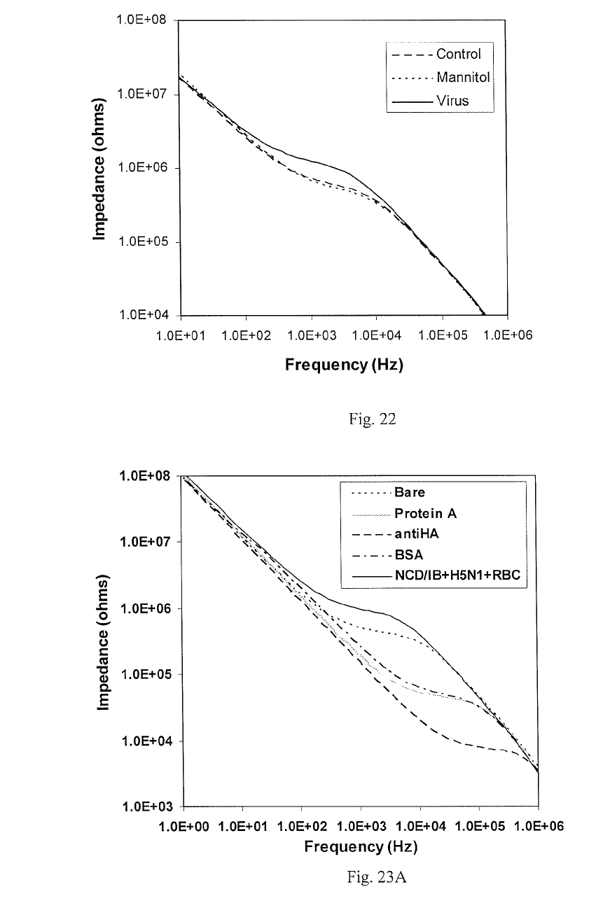

As shown in FIG. 22, avian influenza virus H5N1 was detected using MNACs having the HA-specific polyclonal antibody, which binds to H5N1, coupled thereto. In the figures, “mannitol” refers to buffer alone, and “virus” refers to a sample with the nanoparticle-affinity moiety-virus complex.

### Example 6

### Specificity for Avian Influenza

A combination of Newcastle virus and infectious bronchitis virus was mixed in a 1:1 ratio with H5N1 avian influenza virus. The virus mixture was then optionally incubated with red blood cells and impedance was measured in a microfluidic channel with an embedded interdigitated array microelectrode on which the specific antibody had been immobilized. Impedance measurements were made using an IM-6 impedance analyzer (BAS, West Lafayette, Ind.) with IL-6/THALES software (immobilized antibody impedance biosensor).

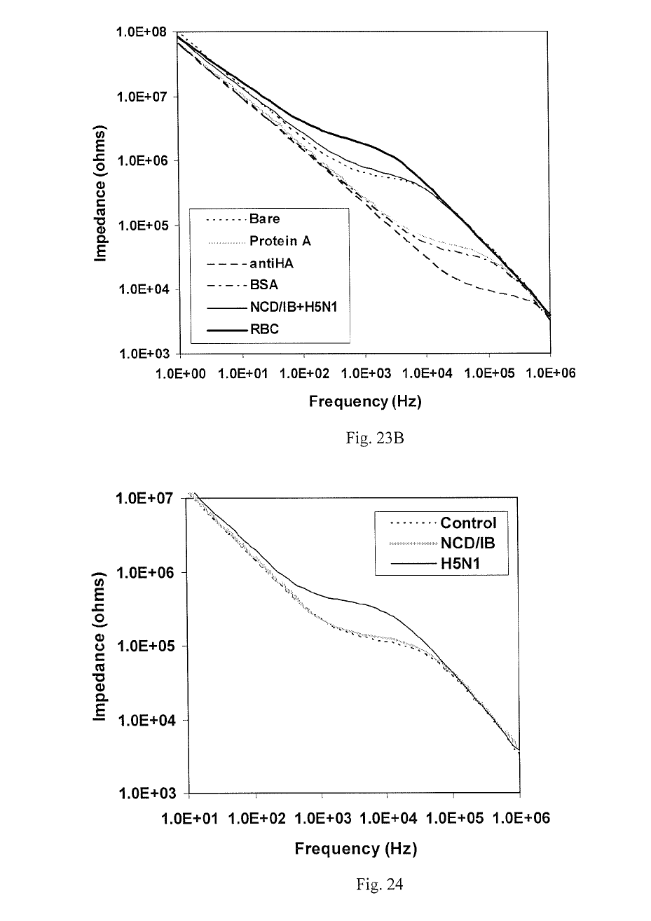

Impedance measurements were made at various stages of coating the biosensor with reagents for antibody immobilization, as indicated. In FIGS. 23A and 23B, “Bare” indicates the impedance of the empty biosensor, “Protein A” is the impedance after addition of Protein A, “anti-HA” indicates the impedance measurement when the biosensor was coated with the antibody, “BSA” indicates the impedance after a wash with PBS-BSA blocking buffer, “NCD/IB+H5N1+RBC” indicates the impedance after addition of the virus mixture and red blood cells (FIG. 23A), “NCD/IB+H5N1” indicates the impedance after addition of the virus mixture but prior to the addition of red blood cells (FIG. 23A), and “RBC” indicates the impedance after the subsequent addition of red blood cells to the biosensor after the viruses had already been added.

In FIG. 23A, the virus mixture was pre-incubated with red blood cells and then passed through the impedance biosensor. The results demonstrate that the mixture was capable of producing a significant change in impedance even in the presence of contaminating viruses.

In FIG. 23B, the virus mixture was passed through the impedance biosensor prior to the addition of red blood cells, and red blood cells were subsequently added after the virus was captured by the biosensor. FIG. 23B demonstrates that the immobilized biosensor was capable of detecting avian influenza in a mixed virus sample, and addition of red blood cells amplified the impedance change. Notably, no signal was detected with Newcastle virus or infectious bronchitis virus even after addition of red blood cells.

### Example 7

### Detection of Virus in Tracheal Swabs after Centrifuge Separation

Tracheal swabs were obtained from chicks using standard procedures and prepared as described above. The swabs were washed into 2 ml Isotonic Dextrose buffer and centrifuged at 2000 rpm for 30 min. The supernatant from this wash was then incubated with 1 ml of a 0.5% suspension (in Isotonic Dextrose with Heparin buffer) of red blood cells for 40 min at 4° C. followed by another round of centrifugation at 1500 rpm for 10 min. The sediment was resuspended in 0.5 ml Isotonic Dextrose buffer and used for impedance analysis.

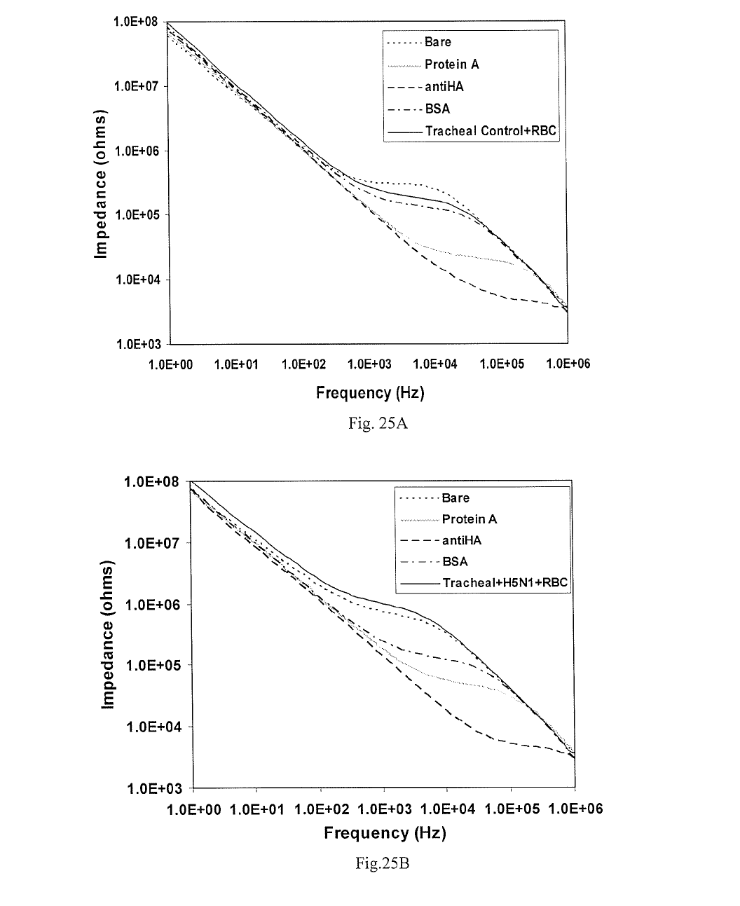

The immobilized impedance biosensor was prepared by coating the biosensor surface with Protein A, followed by anti-HA antibody and blocking with PBS-BSA. The results are shown in FIGS. 25A and 25B. FIG. 25A shows the impedance when no virus was added to the tracheal swab. FIG. 25B shows the impedance when virus was added to the swab. The impedance is significantly increased by the presence of virus in the swab.

### Example 8

### Detection of Virus in Tracheal Swabs after Magnetic Separation

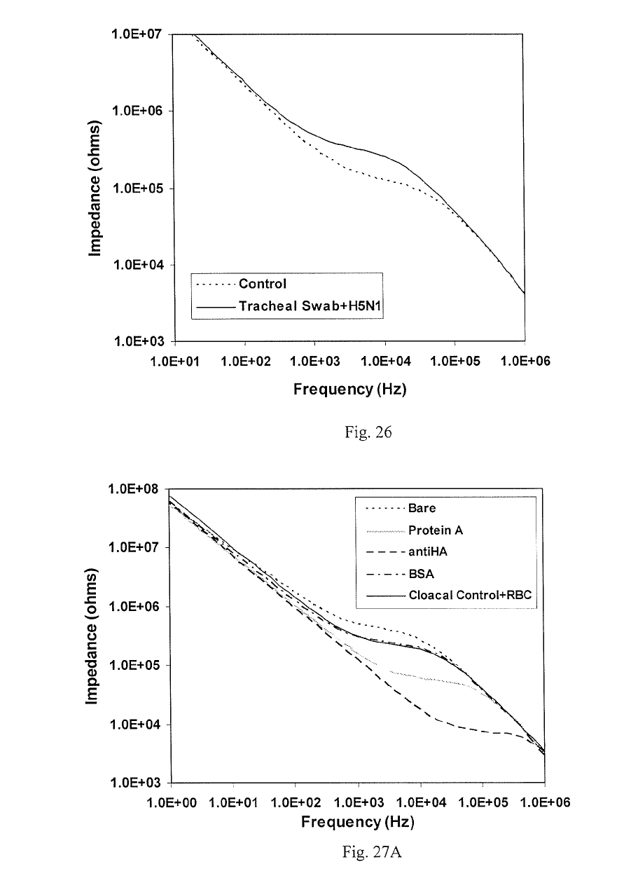

Tracheal swabs were obtained from chicks using standard procedures and prepared as described above. The swabs were washed into 2 ml Isotonic Dextrose with Heparin buffer. The “Control” sample was then incubated with nanoparticle-linked anti-HA antibody complexes produced as described above. The “Tracheal Swab+H5N1” sample was supplemented with H5N1 virus. The tracheal swab was dipped into 10⁵ EID₅₀/ml AIV solution and subsequently washed into 2 ml Isotonic Dextrose buffer. The final concentration of virus is estimated at 10⁴-10⁵ EID₅₀/ml. The resulting virus-nanoparticle-antibody complexes were then magnetically separated as described. The label-free impedance biosensor results are depicted in FIG. 26. The results demonstrate a significant increase in impedance when H5N1 influenza virus is present in the tracheal swab sample as compared to a control swab sample.

### Example 9

### Detection of Virus in Cloacal Swabs after Centrifuge Separation

Cloacal swabs were obtained from chicks using standard procedures and prepared as described above. The swabs were washed into 2 ml Isotonic Dextrose buffer, either with or without virus and centrifuged at 2000 rpm for 30 min. For those samples in which virus was added, the cloacal swab was dipped into 10⁵ EID₅₀/ml AIV solution, and then washed into 2 ml Isotonic Dextrose buffer. The supernatant from this wash was then incubated with 1 ml of 0.5% red blood cells (in Isotonic Dextrose with Heparin buffer) for 40 min at 4° C. followed by another round of centrifugation at 1500 rpm for 10 min. The sediment was resuspended in 0.5 ml Isotonic Dextrose buffer and used for impedance analysis.

The immobilized impedance biosensor was prepared by coating the biosensor surface with Protein A, followed by anti-HA antibody, and finally blocking with PBS-BSA as described above. The results are shown in FIG. 27 and are similar to the results obtained using tracheal swabs. FIG. 27A shows the impedance when no virus was added to the cloacal swab. FIG. 27B shows the impedance when virus was added to the swab. The impedance is significantly increased by the presence of virus in the swab.

### Example 10

### Sensitivity of the Immobilized Impedance Biosensor

Cloacal swabs were obtained from chicks using standard procedures and prepared by exposing the swabs to various concentrations of H5N1 influenza virus. The cloacal swabs were dipped into a series of ten-fold dilutions of AIV solution and subsequently washed into 2 ml Isotonic Dextrose buffer. The swabs were washed into 2 ml Isotonic Dextrose with Heparin buffer and centrifuged at 2000 rpm for 30 min. The supernatant from this wash was then incubated with 1 ml of 0.5% red blood cells (in Isotonic Dextrose with Heparin buffer) for 40 min at 4° C. followed by another round of centrifugation at 1500 rpm for 10 min. The sediment was resuspended in 0.2 ml Isotonic Dextrose with Heparin buffer and used for impedance analysis.

The immobilized impedance biosensor was prepared by coating the biosensor surface with Protein A, followed by anti-HA antibody and blocking with PBS-BSA as described above. The results are shown in FIG. 28. FIG. 28A shows a bar graph of the impedance data obtained at a frequency of 10,400 Hz. The data demonstrate a significant increase in impedance as the virus dilution is decreased from 1/100,000 to 1/10,000, or approximately 10³ EID₅₀/ml. This sensitivity is sufficient for field-testing of infected poultry. FIG. 28B is a graph showing the change in impedance over a range of frequencies for the dilutions of virus.

### Example 11

### Sensitivity of the Label-Free Impedance Biosensor

Cloacal swabs were obtained from chicks using standard procedures and prepared by exposing the swabs to various concentrations of H5N1 influenza virus. The cloacal swabs were dipped into a series of ten-fold dilutions of AIV solution, at 1/100, 1/1000, 1/10000, and 1/100000 final concentrations, and subsequently washed into 2 ml Isotonic Dextrose buffer. The swabs were then incubated with nanoparticle-linked anti-HA antibody complexes produced as described above. The resulting virus-nanoparticle-antibody complexes were then incubated with 1 ml of a 0.5% suspension of red blood cells (in Isotonic Dextrose with Heparin buffer), and subsequently magnetically separated as described above.

The label-free impedance biosensor results are depicted in FIGS. 29A and 29B. FIG. 29A shows a bar graph of the impedance data obtained at a frequency of 4,150 Hz. The data demonstrate a significant increase in impedance as the virus is diluted to 1/100,000 or approximately 10² EID₅₀/ml. This sensitivity is sufficient for field-testing of infected poultry. FIG. 29B is a graph showing the change in impedance over a range of frequencies for the dilutions of virus.

### Example 12

### Detection of Avian Influenza Virus with the Label-Free Impedance Biosensor

Avian influenza H5N1 was also detected using the magnetic nanoparticle-antibody complexes and the label-free impedance biosensor described herein. Briefly, streptavidin-coated nanoparticles were coupled to biotinylated anti-HA antibodies and either H5N1 influenza or a mixture of Newcastle virus and infectious bronchitis virus were added to the nanoparticle-antibody complex. The complexes were passed through the impedance biosensor. The results, depicted in FIG. 24, demonstrate the ability to specifically detect H5N1 virus. The results also show that impedance was not affected by the presence of non-target viruses.

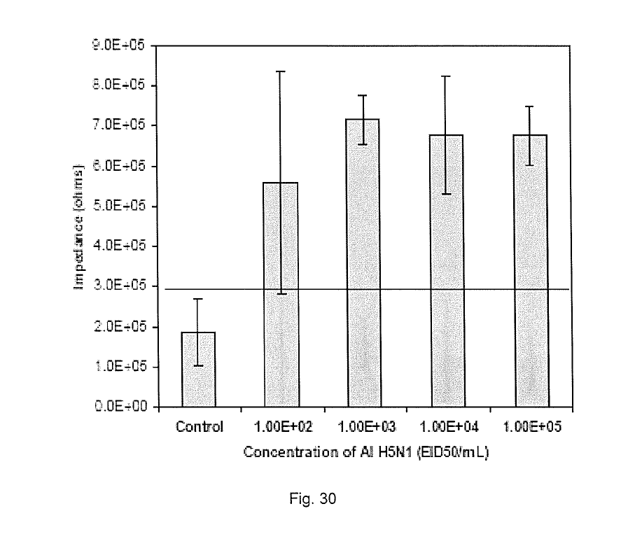

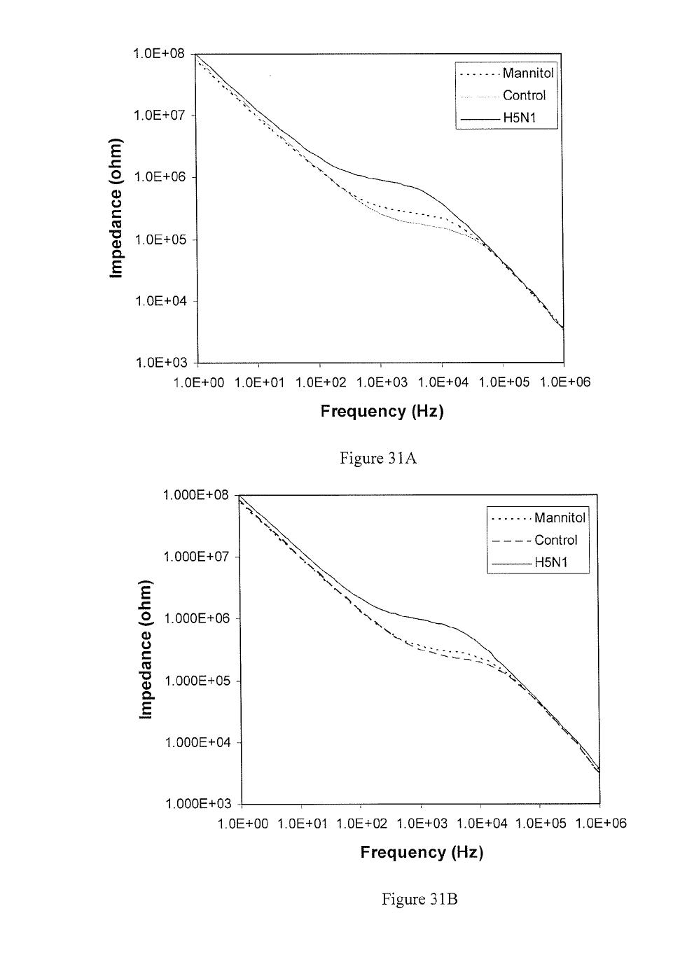

As shown in FIG. 30, a positive signal indicating the presence of the virus was obtained when the concentration of avian influenza virus H5N1 in cloacal swab samples was equal to or more than 100 EID₅₀/mL. FIG. 24 shows that at the frequency of 10 kHz, the impedance of the sample containing avian influenza virus H5N1 is significantly increased compared to either the control (no virus) or the sample containing Newcastle virus and Infectious Bronchitis virus. The results show that the sensitivity is 100-fold more sensitive than the existing antigen-capture ELISA. The maximum change in impedance, from 100 to 800 kΩ, occurred in the range of frequencies from 1 kHz to 100 kHz.

### Example 13

### Detection of Avian Influenza Virus Using Magnetic Nanoparticles and an Immobilized Impedance Biosensor Using a Non-Commercial Antibody

Another set of virus-detection experiments was conducted using a non-commercial polyclonal anti-H5 antibody generated in rabbit. The antibody was raised against recombinant H5 polypeptide (obtained from Protein Science Inc., Meriden, Conn.; A/Vietnam/1203/2004) using standard protocols. Anti-H5 antibodies from the rabbit were purified by ammonium sulphate precipitation, dialyzed against acetate buffer and phosphate buffer and made available for biosensor applications.

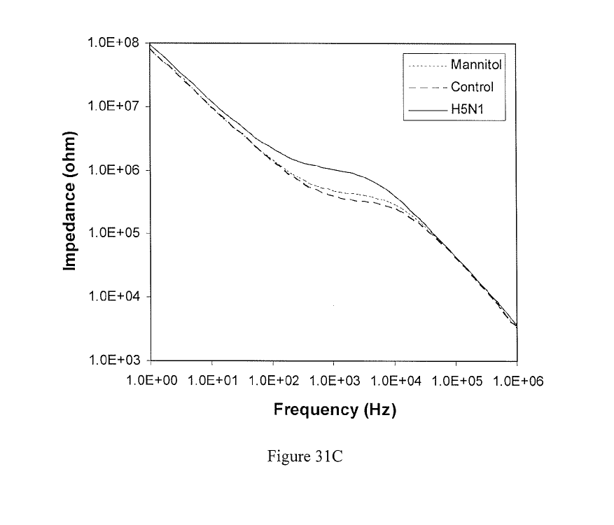

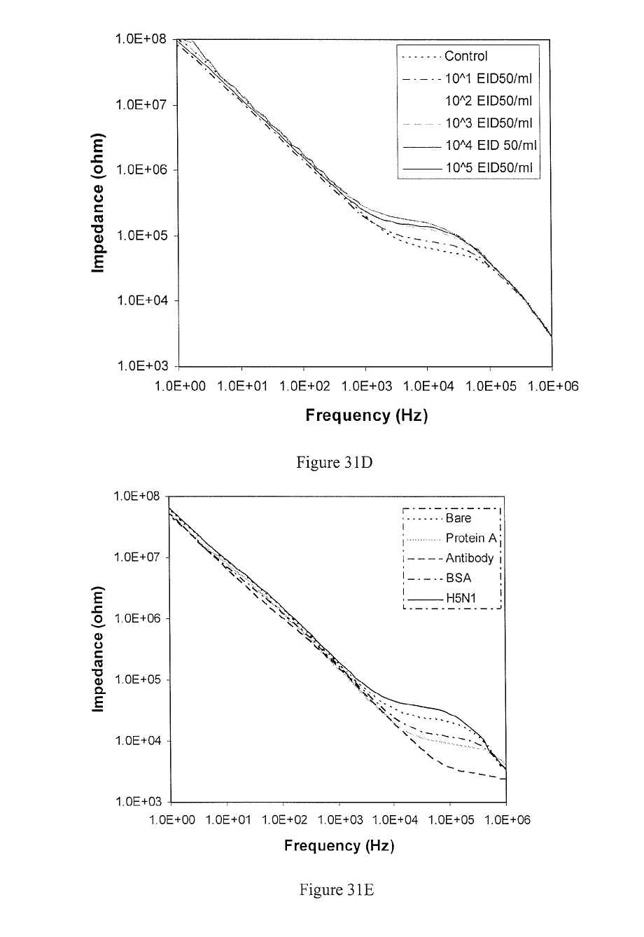

In some experiments (results shown in FIGS. 31A-31D), the antibody was coupled to magnetic nanoparticles, which were combined with AI H5N1 virus particles. The antibody-labeled nanoparticles were combined with varying concentrations of H5N1 virus (or buffer alone as a control) and further processed prior to application onto a label-free biosensor. In other experiments (results shown in FIG. 31E), the IDAM biosensor, which was located in a microfluidic chamber, was coated with the antibody, producing an “immobilized” biosensor.

The anti-H5 antibodies were biotinylated to facilitate attachment to streptavidin-coated nanoparticles. Biotinylation of anti-H5 antibody was performed with EZ-Link Sulfo-NHS Biotinylation Kit according to the supplied instruction. Briefly, 100 μl anti-HA (4.9 mg/ml) was mixed with 3 μl Sulfo-NHS-Biotin solution (10 mM) into 200 μl PBS (10 mM, pH 7.4) and incubated at room temperature for 60 minutes. Then, excess biotin was removed by using Slide-A-Lyzer Dialysis Cassettes. The level of biotin incorporation was measured to be 4 to 5 mole biotin per mole antibody by using HABA assay.

Biotin-labeled anti-H5 antibodies (50 μl) were continuously mixed with streptavidin-coated magnetic nanoparticles (20 μl) in 50 μl PBS at 15 rpm on a variable speed rotator (ATR, Laurel, Md.) for 40 min at room temperature. After antibody immobilization, magnetic nanoparticles were mixed with 100 μl biotin solution (1 mg/ml in PBS) for 20 min to block excess streptavidin present on the surface of magnetic nanoparticles. Excess biotin was washed out by a magnetic separation.

Ten-fold serial dilutions (10⁻², 10⁻³, 10⁻⁴, 10⁻⁵, and 10⁻⁶) of inactivated AI H5N1 virus (dilutions of a solution in which the original titer was 1×10⁷±one log EID₅₀/ml) were suspended in 500 μl buffer (Isotonic Dextrose), and buffer without virus was used as a control. The 500 μl of sample was then mixed with antibody coated nanoparticles for an immunoreaction time of 30 min. After immunoreaction, nanoparticle-virus complexes were washed with 300 μl 0.1 M mannitol solution with an intermittent magnetic separation, and then were resuspended in 150 μl of 0.1 M mannitol solution for impedance measurement.

The antibody was attached to the biosensor using protein A. Firstly, Protein A (1 mg/ml in 10 mM PBS, pH 7.4) was injected into the microfluidic chamber and incubated at room temperature for 1.5-2 hours. Then, the microelectrode was further modified with anti-H5 antibody (245 μg/ml) (incubation for 2.5 hours at room temperature). After blocking with BSA (1% in 10 mM PBS, pH 7.4 and incubate at room temperature for 30 minutes), the chip was read for a sample test. After each immobilization step, the microelectrode was washed with 0.1 M mannitol solution, and impedance measurements were performed.

Impedance measurements were performed using an IM-6 impedance analyzer (BAS, West Lafayette, Ind.) with IM-6/THALES software. For all impedance measurements, a sine-modulated AC potential of 100 mV was applied across the IDAM (interdigitated array microelectrode) and the magnitude of impedance and phase angle were measured for the frequency range from 10 Hz to 1 MHz.

Results of impedance measurements are shown in FIGS. 31A-31E. FIGS. 31A-31C show results from three independent measurements of samples made with 10⁵ EID₅₀/ml virus (“H5N1”) as compared to impedance measurements made with the wash solution (“mannitol”) or with control samples (“control”) containing the Isotonic Dextrose buffer alone. FIG. 31D shows a comparison of impedance measurements performed using samples prepared with the virus dilution series as described above (labeled “10̂1 EID50/ml” through “10̂5 EID50/ml”) as well as the virus-free Isotonic Dextrose buffer (“Control”). FIG. 31E shows impedance measurements made following each step of the antibody-labeling procedure as indicated, on an immobilized biosensor without the use of nanoparticles.

1-52. (canceled)

53. A method of detecting a virus in a starting material comprising:
a) contacting the starting material with a red blood cell, wherein the virus is capable of binding to the red blood cell to form a complex; and
b) detecting the complex with a biosensor, wherein detection of the complex is indicative of the presence of the virus in the starting material.

54. The method of claim 53, wherein step b) further comprises determining the quantity of the virus in the starting material.

55. The method of claim 53, wherein the starting material is selected from the group consisting of a food product, an animal-derived sample, an environmental sample, and a clinical sample.

56. The method of claim 53, wherein the virus is selected from the group consisting of influenza, avian influenza H5N1, infectious bronchitis virus and Newcastle virus.

57. The method of claim 53, wherein step a) further comprises contacting the starting material with an affinity moiety capable of binding to the virus, wherein the affinity moiety is coupled to a magnetic nanoparticle, to form a magnetic target.

58. The method of claim 57, wherein the affinity moiety is an antibody.

59. The method of claim 57, wherein step a) further comprises separating the magnetic target from the starting material.

60. The method of claim 57, wherein the affinity moiety is not attached to the biosensor.

61. The method of claim 57, wherein step b) further comprises
delivering the magnetic target to the biosensor, wherein the biosensor comprises a microfluidic cell having an interdigitated array microelectrode disposed therein; and
detecting the magnetic target with the interdigitated array microelectrode, wherein detection of the target is indicative of the presence of the contaminant in the starting material.

62. The method of claim 61, wherein step b) further comprises measuring impedance of the interdigitated array microelectrode.

63. The method of claim 61, wherein step b) further comprises determining the quantity of the virus in the starting material.

64. The method of claim 61, wherein the starting material is selected from the group consisting of a food product, an animal-derived sample, an environmental sample, and a clinical sample.

65. The method of claim 61, wherein the virus is selected from the group consisting of influenza, avian influenza H5N1, infectious bronchitis virus and Newcastle virus.
